(19) United States
(12) Patent Application Publication
(10) Pub. No.: US 2011/0300124 A1
(43) Pub. Date: Dec. 8, 2011

(54) SOAKING AGENT FOR THE TREATMENT OF HEMORRHOIDS AND METHOD OF USE

(76) Inventor: Joseph P. Thornton, Las Vegas, NV (US)

(21) Appl. No.: 12/547,193

(22) Filed: Aug. 25, 2009

(51) Int. Cl. A61K 38/46; A61P 43/00

(52) U.S. Cl. 424/94.65

(57) ABSTRACT

A natural gel/ointment for the treatment of hemorrhoids. Bromelain is one primary active ingredient in the gel/ointment. A second active ingredient in the form of Witch Hazel may be combined with the Bromelain. Supplemental ingredients include alcohol, de-ionized water, Butylene Glycol, PEG-40 Hydrogenated Castor Oil, Hydroxyethylcellulose, Eucalyptus Globulus Oil, Aloe Barbadensis Leaf Extract, Chamomilla Rectutita Flower Extract, Camellia Sinensis Leaf Extract, Avena Sativa Kernel Flour, Benzophenone-4, Disodium EDTA, Sodium Citrate, Sodium Hydroxide, Citric Acid, Caprylyl Glycol, Chlorphenesin and Methylisothiazolinone. Based on case studies the gel/ointment outperforms other hemorrhoid medications.

### RELATED APPLICATIONS

This application is related to co-pending U.S. application Ser. No. 12/______ and U.S. application Ser. No. 12/______ , both of which are filed herewith and incorporated herein in their entirety.

### FIELD OF THE INVENTION

The embodiments of the present invention relate to a natural, soaking agent for the treatment of hemorrhoids.

### BACKGROUND

Hemorrhoids are swollen veins in the anal canal. Millions of people suffer from hemorrhoids and a majority of adults will suffer from hemorrhoids at one point in their life. Treatments for hemorrhoids range from surgery to topical ointments. Surgery includes rubber band ligation, cryosurgery and surgical hemorrhoidectomy. Non-surgical procedures include the application of heat, use of lasers or electric current to create scar tissue, or sclerotheraphy. Medicinal treatments include ointments and creams such as Preparation H®, medicated wipes and the like. Despite the various treatments, hemorrhoids continue to flourish.

Thus, there exists a need for natural treatment to more reliably and efficiently treating hemorrhoids.

### SUMMARY

Accordingly, one embodiment of the present invention is a natural, soaking agent including at least one anti-inflammatory agent. In one embodiment, the soaking agent includes Bromelain as the anti-inflammatory agents. Other ingredients are compounded with the Bromelain to form a soaking agent as the delivery vehicle.

In one embodiment, one tablespoon of the soaking agent is placed into warm, running bath water and the subject individual soaks for 15-20 minutes. In one embodiment, the soaking agent is in a granular or powder form such that it dissolves in the bath water.

Other variations, embodiments and features of the present invention will become evident from the following detailed description, drawings and claims.

### BRIEF DESCRIPTION OF THE DRAWINGS

FIG. 1 illustrates an ingredient list for a natural soaking agent according to the embodiments of the present invention; and

FIG. 2 illustrates a kit container designed to hold a container of the soaking agent along with the tablets and gel/ointment as described in the referenced co-pending applications.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

A natural treatment for hemorrhoids has benefits for both the individual with the ailment and society as a whole. The benefit to society includes a decreased need for surgery, prescription drugs and other unnecessary unnatural treatments. The individual suffering from hemorrhoids benefits by being able to utilize an easy-to-use, soaking agent. Over-the-counter topical Preparation H® utilizes mineral oil (protectant), petrolatum (protectant), Phenylephrine (vasoconstrictor) and shark liver oil (protectant). Over-the-counter topical Tucks® utilizes a combination of mineral oil (protectant), Pramoxine HCL (pain relief) and Zinc Oxide (protectant). Over-the-counter topical Balneol® is essentially a moisturizer. Therefore, the active ingredients in most over-the-counter topical applications are designed primarily to protect and treat pain in the area affected by hemorrhoids but do not effectively treat or prevent the hemorrhoids. Prescription drugs, like topical Analpram, utilize Pramoxine HCL (pain relief) and Hydrocortisone (steroidal anti-inflammatory) but still fail to treat hemorrhoids adequately. Another benefit to the over-the-counter natural soaking agent is that is can be used in conjunction with the tablet described in co-pending patent application Ser. No. 12/______ and/or the topical ointment/gel described in co-pending patent application Ser. No. 12/______ . Any of the treatments alone or in combination facilitate the avoidance of expensive prescription drugs. In many instances today, patients are not able to afford prescription drugs. This is true for those having insurance given large deductibles and uncovered prescriptions.

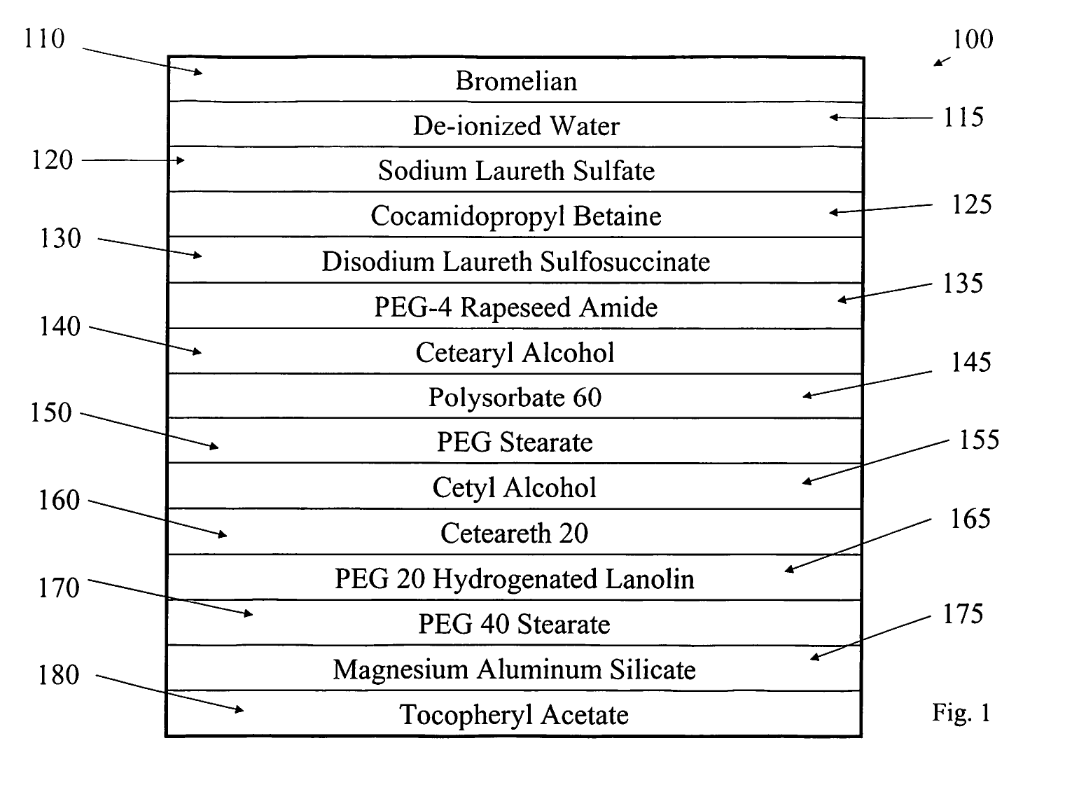

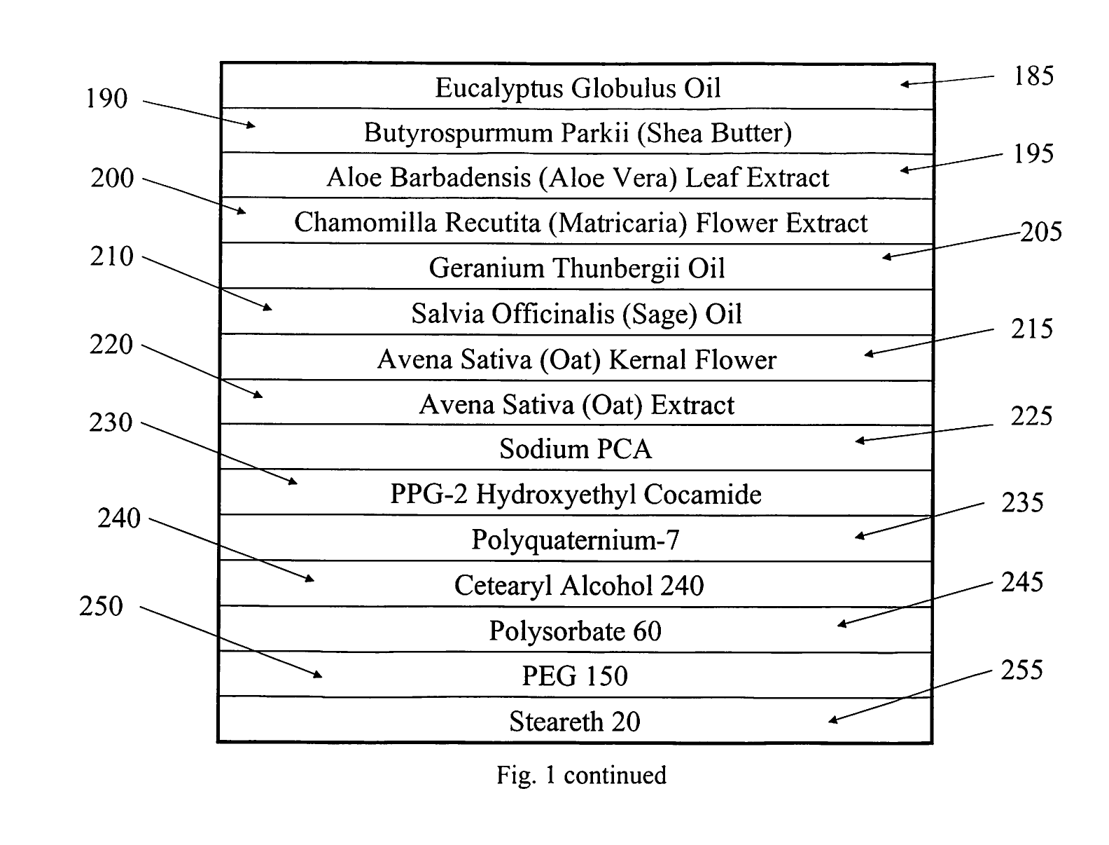

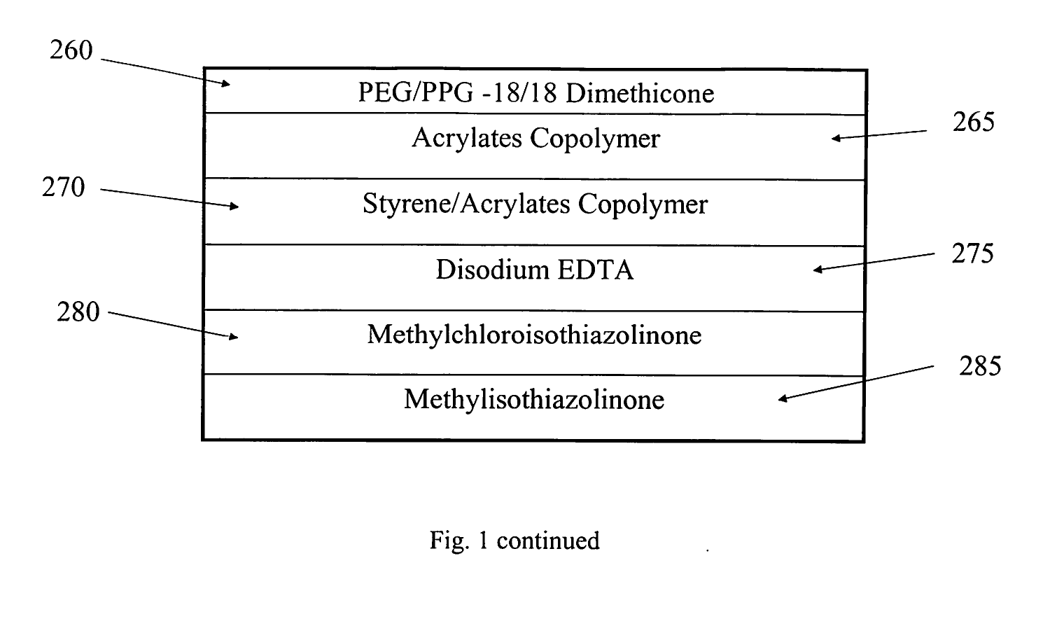

FIG. 1 depicts an ingredient list 100 for a first embodiment of the present invention. The primary active ingredient is Bromelain 110. The Bromelain 110 has an anti-inflammatory effect. Bromelain is a mixture of protein-digesting (proteolytic) enzymes found in pineapples. Bromelain, which is derived from the stem and juice of the pineapple, was first isolated from the pineapple plant in the late 1800s.

In a first embodiment, as depicted in the ingredient list 100 the soaking agent according to the embodiments of the present invention comprises secondary ingredients including De-ionized Water 115, Sodium Laureth Sulfate 120, Cocamidopropyl Betaine 125, Disodium Laureth Sulfosuccinate 130, PEG-4 Rapeseed Amide 135 (and) Cetearyl Alcohol 140 (and) Polysorbate 60 145 (and) PEG Stearate 150 (and) Cetyl Alcohol 155 (and) Ceteareth 20 160 (and) PEG 20 Hydrogenated Lanolin 165 (and) PEG 40 Stearate 170, Magnesium Aluminum Silicate 175, Tocopheryl Acetate 180, Eucalyptus Globulus Oil 185, Butyrospurmum Parkii (Shea Butter) 190, Aloe Barbadensis (Aloe Vera) Leaf Extract 195, Chamomilla Recutita (Matricaria) Flower Extract 200, Geranium Thunbergii Oil 205, Salvia Officinalis (Sage) Oil 210, Avena Sativa (Oat) Kernel Flower 215, Avena Sativa (Oat) Extract 220, Sodium PCA 225, PPG-2 Hydroxyethyl Cocamide 230, Polyquaternium-7 235, Cetearyl Alcohol 240 (and) Polysorbate 60 245 (and) PEG 150 250 (and) Steareth 20 255, PEG/PPG-18/18 Dimethicone 260, Acrylates Copolymer 265, Styrene/Acrylates Copolymer 270, Disodium EDTA 275, Methylchloroisothiazolinone 280 and Methylisothiazolinone 285. Those skilled in the art will recognize that other ingredients having the same properties may be used in place of, or in addition to, the aforementioned secondary ingredients. Also, certain of the listed secondary ingredients may be removed while maintaining the effectiveness of the soaking agent. The ingredients are combined into a granules or powder using conventional manufacturing and compounding techniques.

In one embodiment, the soaking agent in accordance with the specifications listed above, is used in a bath of warm water for 15-20 minutes as needed to allow the subject to feel relief from hemorrhoid symptoms

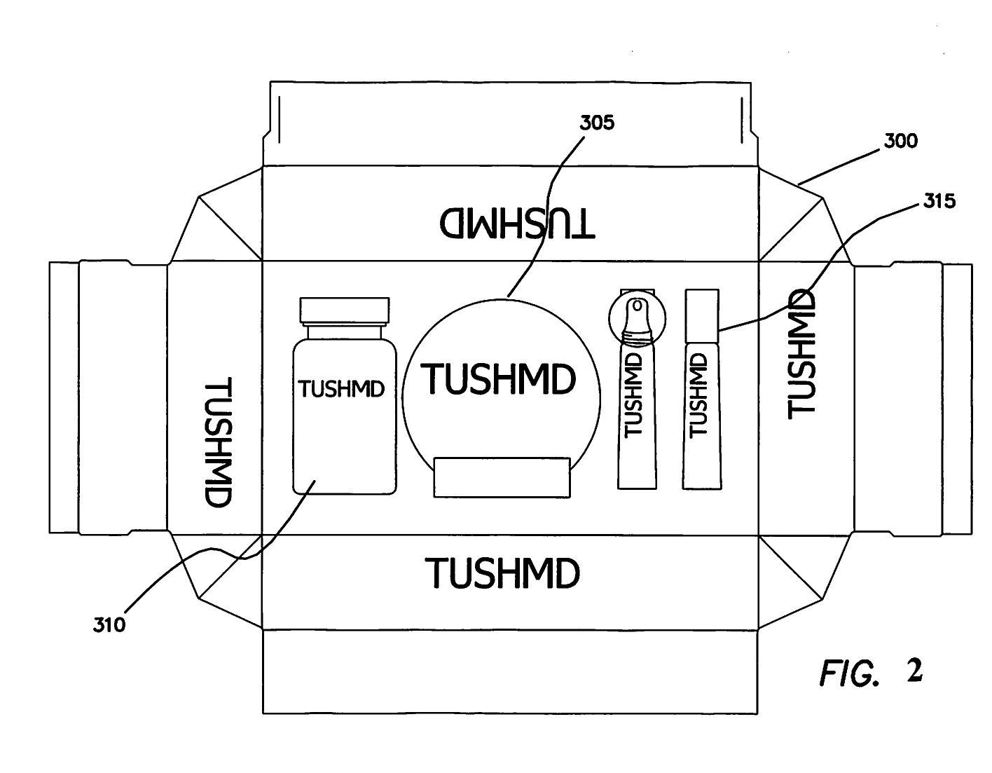

FIG. 2 shows a form 300 for constructing a box to hold a kit comprising individual containers for each of the soaking agent 305, tablets 310 (described in co-pending patent application Ser. No. 12/______) and gel/ointment 315 (described in co-pending patent application Ser. No. 12/______). Separately or alone, the soaking agent, tablets and gel/ointment effectively treat hemorrhoids. The treatment protocols for each delivery vehicle remain the same when each is used in combination as when used individually. In other words, all three treatment protocols can be used simultaneously without any negative side effects.

Although the invention has been described in detail with reference to several embodiments, additional variations and modifications exist within the scope and spirit of the invention as described and defined in the following claims.

I claim:

1. A soaking agent for the treatment of hemorrhoids comprising:
bromelain; and
one or more ingredients in combination with said bromelain to form granules or powder.

2. The soaking agent of claim 1 wherein said one or more ingredients comprise one or more of the following: De-ionized Water, Sodium Laureth Sulfate, Cocamidopropyl Betaine, Disodium Laureth Sulfosuccinate, PEG-4 Rapeseed Amide, Cetearyl Alcohol, Polysorbate 60, PEG Stearate, Cetyl Alcohol, Ceteareth 20, PEG 20 Hydrogenated Lanolin, PEG 40 Stearate, Magnesium Aluminum Silicate, Tocopheryl Acetate, Eucalyptus Globulus Oil, Butyrospurmum Parkii (Shea Butter), Aloe Barbadensis (Aloe Vera) Leaf Extract, Chamomilla Recutita (Matricaria) Flower Extract, Geranium Thunbergii Oil, Salvia Officinalis (Sage) Oil, Avena Sativa (Oat) Kernel Flower, Avena Sativa (Oat) Extract, Sodium PCA, PPG-2 Hydroxyethyl Cocamide, Polyquaternium-7, Cetearyl Alcohol, Polysorbate 60, PEG 150, Steareth 20, PEG/PPG-18/18 Dimethicone, Acrylates Copolymer, Styrene/Acrylates Copolymer, Disodium EDTA, Methylchloroisothiazolinone and Methylisothiazolinone.

3. A kit comprising:
a soaking agent comprising:
bromelain; and
one or more ingredients in combination with said bromelain to form granules or powder;
an orally ingested tablet comprising:
bromelain; and
a laxative; and
a topical medication comprising:
bromelain; and
one or more ingredients in combination with said bromelain to form a gel or ointment.

4. The kit of claim 3 wherein the orally ingested tablet further comprises hamamelis virginiana.

5. The kit of claim 3 wherein the gel/ointment further comprises hamamelis virginiana.

6. The kit of claim 3 wherein said laxative is Psyllium-based.

7. The kit of claim 3 wherein said laxative is Psyllium husk powder.

8. The kit of claim 3 wherein said orally ingested tablet further comprises one or more of the following ingredients: Dicalcium Phosphate, Cellulose, Croscarmellose Sodium, Stearic Acid and Silicon Dioxide.

9. The kit of claim 3 wherein said one or more ingredients of the gel/ointment comprise one or more of the following: alcohol, de-ionized water, Butylene Glycol, PEG-40 Hydrogenated Castor Oil, Hydroxyethylcellulose, Eucalyptus Globulus Oil, Aloe Barbadensis Leaf Extract, Chamomilla Rectutita Flower Extract, Camellia Sinensis Leaf Extract, Avena Sativa Kernel Flour, Benzophenone-4, Disodium EDTA, Sodium Citrate, Sodium Hydroxide, Citric Acid, Caprylyl Glycol, Chlorphenesin and Methylisothiazolinone.

10. The kit of claim 3 wherein said one or more ingredients of the soaking agent comprise one or more of the following: De-ionized Water, Sodium Laureth Sulfate, Cocamidopropyl Betaine, Disodium Laureth Sulfosuccinate, PEG-4 Rapeseed Amide, Cetearyl Alcohol, Polysorbate 60, PEG Stearate, Cetyl Alcohol, Ceteareth 20, PEG 20 Hydrogenated Lanolin, PEG 40 Stearate, Magnesium Aluminum Silicate, Tocopheryl Acetate, Eucalyptus Globulus Oil, Butyrospurmum Parkii (Shea Butter), Aloe Barbadensis (Aloe Vera) Leaf Extract, Chamomilla Recutita (Matricaria) Flower Extract, Geranium Thunbergii Oil, Salvia Officinalis (Sage) Oil, Avena Sativa (Oat) Kernel Flower, Avena Sativa (Oat) Extract, Sodium PCA, PPG-2 Hydroxyethyl Cocamide, Polyquaternium-7, Cetearyl Alcohol, Polysorbate 60, PEG 150, Steareth 20, PEG/PPG-18/18 Dimethicone, Acrylates Copolymer, Styrene/Acrylates Copolymer, Disodium EDTA, Methylchloroisothiazolinone and Methyl isothiazolinone.
